(19) United States
(12) Patent Application Publication
(10) Pub. No.: US 2003/0161902 A1
(43) Pub. Date: Aug. 28, 2003

(54) Increased lifespan formulation

(75) Inventor: Kelvin Winston Duncan, Christchurch (NZ)

Correspondence Address: GREER, BURNS & CRAIN, 300 S WACKER DR, 25TH FLOOR, CHICAGO, IL, 60606, US

(73) Assignee: Enzo Natraceuticals Limited.

(21) Appl. No.: 10/327,760

(22) Filed: Dec. 23, 2002

(30) Foreign Application Priority Data
Dec. 24, 2001 (NZ) NZ 516 366

(51) Int. Cl. A61K 35/78; A61K 31/7048; A61K 31/353

(52) U.S. Cl. 424/770; 514/27; 514/456

(57) ABSTRACT

A composition including a dose of flavonoids extracted from pine bark extract is described that decreases the rate of senescence of a mammal after onset of senescence. The same compound also increases mammal lifespan and increases neuromuscular performance after onset of senescence. Methods of treatment and use of this composition are also described. The composition has the advantage of being a useful general aging remedy to avoid many common age related decreases in performance.

### BACKGROUND

The present invention relates to a composition for use to decrease the rate of senescence in mammals after the onset of senescence. The present invention also relates to compositions for increasing the life span of a mammal and additionally, to increase the level of neuromuscular performance of a mammal after senescence begins.

The potential use of dietary supplements for protection against the effects of oxidative stress and the progression of degenerative diseases and aging has been the subject of an increasing number of studies during the past two decades.

The “Free Radical Theory” of disease and aging holds that deleterious metabolic and cellular events caused by free radical reactions are responsible for the phenomena of disease and aging¹⁻⁹. The fact that many important or essential macromolecules of the body are damaged by free radical events has been repeatedly demonstrated¹⁰. Thus aging and disease are seen as an imbalance in the pro-oxidant/antioxidant balance in the body. However, not all free radical reactions in the body are harmful; some are entirely natural and are necessary for the correct functioning of many metabolic processes¹¹. Furthermore, there is an increase in free radical formation as a consequence of disease rather than as a cause of the disease¹². This “consequence not cause” hypothesis has cast doubt on the role of antioxidants in preventing disease.

In theory, pro-oxidant/antioxidant imbalances maybe corrected, at least in part, by increasing the consumption of antioxidants. Rodents have been used for trials to show this trend. Rodents are advantageous test mammals as they share a similar genome to humans and have long been used to test potential drugs³⁰.

One example of correcting a pro-oxidant/antioxidant imbalance is that described in pending application NZ516367, incorporated herein by reference. In this application, DNA and protein damage from oxidative stress is described.

Generally speaking, rodent trials examining the effect of dietary supplementation using antioxidants on demographic performance have shown results that have been somewhat inconclusive^{13,17}, ineffective^{14,15,16,18} or even negative²². Increases in mean survivorship, but not maximum life span, have been reported when vitamin E¹⁹, vitamin C²⁰, and Ginko biloba extract²¹ were administered.

One particular publication³¹ does describe a trial where an increase in maximum lifespan is found for a rodent study. The increase was found by feeding mice a supplement containing a synthetic reducing agent, 2-mercaptoethanol. Mercaptoethanol compounds have a sulphahydryl anti-oxidant action. This substance however has the negative effects of being toxic, not containing naturally occurring flavonoids and also has a very unpleasant odor. In particular it would appear that the study did not eliminate caloric restriction as a variable from the trial. As a result, the increased life span shown was more likely due to other factors besides supplementation by 2-mercaptoethanol.

For a successfull trial the choice of antioxidant is important. Highly specific or synthetic antioxidants are open to two possible handicaps:

(1) They may be a racemic mixture of stereoisomers which may lead to confounded results through deleterious effects induced by isomers of the wrong chirality, as demonstrated with synthetic vitamin E;

(2) Single chemical species of free radical scavengers have been shown to have a quite narrow specificity for particular free radical chemical species.

Other hypotheses have been suggested to account for increased survivorship of mammals. In particular, dietary restriction has been shown to prolong life, though at the expense of body size and fecundity.

It is therefore an object of the present invention to provide a composition that decreases the rate of senescence of a mammal after onset of senescence.

It is a further object of the present invention to provide a composition that increases the life span of a mammal.

It is a further object of the present invention to provide a composition that maintains or increases neuromuscular performance of the mammal after the onset of senescence.

It is a further object of the present invention to address the foregoing problems or at least to provide the public with a useful choice.

All references, including any patents or patent applications, cited in this specification are hereby incorporated by reference. No admission is made that any reference constitutes prior art. The discussion of the reference states what their authors assert, and the applicants reserve the right to challenge the accuracy and pertinency of the cited documents. It will be clearly understood that, although a number of prior art publications are referred to herein, this reference does not constitute an admission that any of these documents forms parts of the common general knowledge in the art in any country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description, which is given by way of example only.

### SUMMARY

In accordance with one aspect of the present invention there is provided a composition including a dose of flavonoids extracted from pine bark that has at least one mode of action selected from the group including: decreasing the rate of senescence of a mammal after onset of senescence; increasing the life span of a mammal; maintaining neuromuscular performance of a mammal after onset of senescence; increasing neuromuscular performance of a mammal after onset of senescence; and combinations thereof.

The above composition has been found by the applicant to be particularly advantageous in delaying the rate of senescence and its related symptoms in mammals. Trials completed by the applicant on mice indicate a reduction in the rate of senescence after onset by as much as 37% for mice fed with a regular dose of pine bark extract. This in itself has the implication of an improved quality of life for mammals after the onset of senescence.

Further results indicate an increase in life span by 8 to 17%. Further results also show that the proportion of mice surviving longer is 80% higher for those taking a regular dose of pine bark extract. This combination of results and the close phylogenetic relationship between mice and humans³⁰ show that this composition can be used to effectively increase the lifespan of a human.

Further tests completed on neuromuscular performance also confirm the reduction in the rate of senescence after onset. Decline in neuromuscular performance is a common symptom of the aging process and elderly mice fed with pine bark extract show significantly improved performance (nine-fold improvement) in neuromuscular performance as measured via a balance test.

In preferred embodiments, the composition as described above is delivered to the mammal as a regular daily dosage. The preferred dose rate for the composition is between 0.5 and 100 mg/kg of body mass per day for the purpose of extension of life. It is understood by the applicant that dose rates may vary between these levels depending on the metabolism level of the mammal and other biochemical factors, such as seasonal dietary requirements. More preferably, the dose is 5 mg/kg of body mass per day.

It will also be appreciated by those skilled in the art that a dosage greater than that of 100 mg/kg is also possible. Pine bark extract is non-toxic and has a naturally occurring source that is a flavonoid-rich substance. Higher doses would not produce any toxic reactions to the subject and may in fact be advantageous for some subjects that require additional oxidative treatment for reasons described above.

Preferably, the pine bark extract used in the above composition includes primarily flavonoid compounds and associated compounds.

Most preferably the pine bark extract composition as described above is an extract which exhibits antioxidant behavior in vivo.

Preferably, the pine bark extract used in the above composition is sourced from the bark of Pinus radiata (the Monterey pine or radiata pine).

Most preferably, the pine bark extract (from Pinus radiata bark), is extracted using a water-based process. One example process is that of NZ329658/U.S. Pat. No. 5,968,517, incorporated herein by reference. It is a complex mixture of mainly flavonoids with some non-flavonoid compounds. Phenolic compounds of Pinus radiata bark include catechin, epicatechin, quercetin, dihydroquercetin, taxifolin, phenolic acids, and procyanidin dimers, trimers, oligomers and polymers formed from catechin and epicatechin. The extract is non-toxic to mice and humans, and it has a broad spectrum of action against a wide variety of free radical events.

Optionally, the composition, substantially as described above, further includes other anti-oxidant active components. These include vitamin C, vitamin E, Ginko biloba, and other known therapeutically active compounds.

In a further option the composition, substantially as described above, is also formulated using components selected from the group including; fillers; excipients; modifiers; humectants; stabilizers; emulsifiers; and other known formulation components.

Preferably, the composition, substantially as described above, is administered in a form selected from the group including: a tablet; a capsule; a suppository; an injection; a suspension; a drink; a tonic; a syrup; a powder; an ingredient in solid foods; an ingredient in liquid foods; and combinations thereof. Most preferably, the composition is administered orally as a powder mixed with food.

According to a;further aspect of the invention, there is provided the use of a composition including a;dose of flavonoids extracted from pine bark, wherein the composition is administered to a mammal to have at least one mode of action on the mammal selected from the group including: to reduce the rate of senescence of a mammal after onset of senescence; to increase the life span of a mammal; to maintain the neuromuscular performance of a mammal after the onset of senescence; to increase the neuromuscular performance of a mammal after the onset of senescence; and combinations thereof.

According to a further aspect of the invention, there is provided a method of treatment of a mammal by administration of a composition including a dose of flavonoids extracted from pine bark to a mammal, to have at least one mode of action on the mammal selected from the group including: to reduce the rate of senescence of a mammal after onset of senescence; to increase the life span of a mammal; to maintain the neuromuscular performance of a mammal after the onset of senescence; to increase the neuromuscular performance of a mammal after the onset of senescence; and combinations thereof.

It can be seen from the above description that by administration of a composition containing Pinus radiata bark extract, the rate of senescent decline (after onset) can be decreased, the lifespan can be increased and the neuromuscular performance of a mammal can be improved, thus providing a general anti-aging treatment.

### BRIEF DESCRIPTION OF DRAWINGS

Further aspects of the present invention will become apparent from the ensuing description, which is given by way of example only and with reference to the accompanying drawings in which:

FIG. 1 Is a graph of results on the use of the composition of the present invention showing the onset and rate of senescence for a control dose;

FIG. 2 Is a graph of results on the use of the composition of the present invention showing the onset and rate of senescence for a 5 mg/kg dose;

FIG. 3 Is a graph of results on the use of the composition of the present invention showing the onset and rate of senescence for a 21 mg/kg dose;

The rate of senescence described above is summarized below in FIG. 4:

FIG. 4 Is a graph of results on the use of the composition of the present invention showing the rate of senescence with dose for a control; 5 mg/kg; 21 mg/kg and 100 mg/kg doses;

Also, a reflection of the lowered rate of senescence was the increased lifespan of the mice by between 12% and 27% in dosed animals and the improved neuromuscular performance of the mice. This analysis is shown in FIGS. 5 to 8:

FIG. 5 Is a graph of results on the use of the composition of the present invention showing the survivorship curves with dose;

FIG. 6 Is a graph of results on the use of the composition of the present invention showing the mean life span with dose;

FIG. 7 Is a graph of results on the use of the composition of the present invention showing the age specific survivorship; and

FIG. 8 Is a graph of results on the effect of dose on neuromuscular performance.

### DETAILED DESCRIPTION

The invention will now be further described with reference to more detailed examples.

With reference to the attached drawings, the methodology and process is described below:

A first experiment was conducted on same aged white mice of the Swiss Outbreed and BALB-C strains. “Weaners” were young animals aged 110 days, obtained shortly after weaning and independent from their mothers. “Geriatric” mice, about 470 days old, were obtained after having served as breeding stock for their productive lives. Both sexes were investigated separately.

Preferably, the pine bark extract used in the present composition includes primarily flavonoid compounds and associated compounds. Most preferably the pine bark extract composition as described above is an extract which exhibits antioxidant behavior in vivo. Preferably, the pine bark extract used in the above composition is sourced from the bark of Pinus radiata (the Monterey pine or radiata pine).

Most preferably, the pine bark extract (from Pinus radiata bark), is extracted using a water-based process. One example process is that of NZ329658/U.S. Pat. No. 5,968,517, incorporated herein by reference. It is a complex mixture of mainly flavonoids with some non-flavonoid compounds. Phenolic compounds of Pinus radiata bark include catechin, epicatechin, quercetin, dihydroquercetin, taxifolin, phenolic acids, and rprocyanidin dimers, trimers, oligomers and polymers formed from catechin and epicatechin. The extract is non-toxic to mice and humans, and it has a broad spectrum of action against a wide variety of free radical events.

Optionally, the composition, substantially as described above, further includes other anti-oxidant active components. These include vitamin C, vitamin E, Ginko biloba, and other known therapeutically active compounds.

In a further option the composition, substantially as described above, is also formulated using components selected from the group including; fillers; excipients; modifiers; humectants; stabilizers; emulsifiers; and other known formulation components.

The mice were divided up into groups and fed varying doses of pine bark extract manufactured in accordance with the method described in NZ329658/U.S. Pat. No. 5,968,517 (incorporated herein by reference) as shown in Table 1 below:

TABLE 1
Design of the experiment and number of animals in each trial

| | | | DOSE | |
|---|---|---|---|---|
| TRIAL | SEX | STRAIN | (mg/kg equivalent) | NUMBER |
| Weather | F | Swiss outbred | 0 | 5 |
| Weaner | F | Swiss outbred | 1 | 5 |
| Weaner | F | Swiss outbred | 5 | 5 |
| Weaner | F | Swiss outbred | 21 | 5 |
| Weaner | F | Swiss outbred | 100 | 5 |
| Weaner | F | Swiss outbred | 0 | 5 |
| Weaner | M | Swiss outbred | 1 | 5 |
| Weaner | M | Swiss outbred | 5 | 5 |
| Weaner | M | Swiss outbred | 21 | 5 |
| Weaner | M | Swiss outbred | 100 | 5 |
| Geriatric | M | Swiss outbred | 0 | 25 |
| Geriatric | F | Swiss outbred | 21 | 25 |
| Geriatric | F | BALV-C | 0 | 24 |
| Geriatric | F | BALB-C | 5 | 20 |
| Weaner | F | Swiss outbred | 0 | 10 |
| Weaner | F | Swiss outbred | 1 | 10 |
| Weaner | F | Swiss outbred | 100 | 10 |
| Weaner | M | Swiss outbred | 0 | 10 |
| Weaner | M | Swiss outbred | 1 | 10 |
| Weaner | M | Swiss outbred | 100 | 10 |
| | | TOTAL | | 204 |

It should be noted that other types of pine bark extract can also be used to achieve the same purpose.

The experimental mice were fed ad libitum on Archer's mouse food pellets dosed with measured doses of pine bark extract manufactured as above. Preferably, the present composition is administered in a form selected from the group including: a tablet; a capsule; a suppository; an injection; a suspension; a drink; a tonic; a syrup; a powder; an ingredient in solid foods; an ingredient in liquid foods; and combinations thereof, all of which are considered equivalent. Most preferably, the composition is administered orally as a powder mixed with food. The antioxidant composition of the food was as follows (Table 2 below):

TABLE 2
Antioxidant and pheolic composition of the mouse food and pine bark
extract

| | | RELATIVE | |
|---|---|---|---|
| | EC50 | ANTIOXIDANT | TOTAL PHENOL |
| SAMPLE | (μg/ml) | ACTIVITY | (μG/ML) |
| Mouse pellet | 6.7 | 1 | 0.22 ± 0.33 |
| Vitamin C | 3.79 | 1.76 | — |
| Pine Bark Extract | 0.28 | 23.9 | 9.64 ± 0.66 |

TABLE 3
Typical gel permeation chromatography fractionation of pine bark
extract manufactured as above quoted as percentages of the total phenolic
compounds present in the extract.

| | | Olig- | |
|---|---|---|---|
| Carbohy- | Mono- | omeric | Poly- |
| drates, Esters & | eric to Trimeric | Proantho- | meric |
| Otganic Acids | Proanthocyanidins | cyanidins | Proanthocyanadins |
| 15% | 22% | 25% | 38% |

The doses were arranged in a logarithmic series based on doses of 0, 1, 5, 21 and 100 mg of pine bark extract/kg of body mass. Because mice did not eat all the proffered food and wasted a portion of their ration, the dose was adjusted to compensate for this wastage so that they received the correct dose in the food they did consume. The preferred dose rate for the composition is between 0.5 and 100 mg/kg of body mass per day for the purpose of extension of life. It is understood by the applicant that dose rates may vary between these levels depending on the metabolism level of the mammal and other biochemical factors, such as seasonal dietary requirements. More preferably, the dose is 5 mg/kg of body mass per day.

It will also be appreciated by those skilled in the art that a dosage greater than that of 100 mg/kg is also possible. Pine bark extract is non-toxic and has a naturally occurring source that is a flavonoid-rich substance. Higher doses would not produce any toxic reactions to the subject and may in fact be advantageous for some subjects that require additional oxidative treatment for reasons described above.

A statistical analysis of the results showed that dietary supplementation was found to have profound effects on demographic performance of the mice.

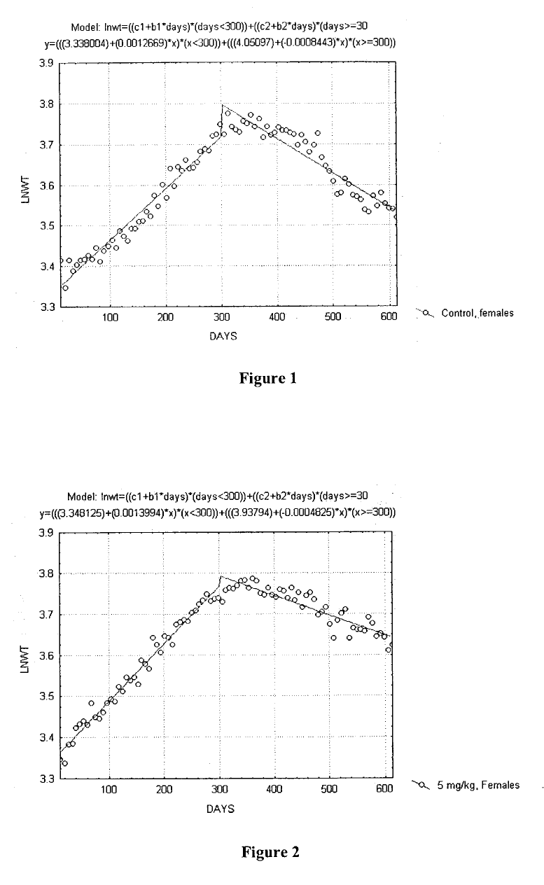

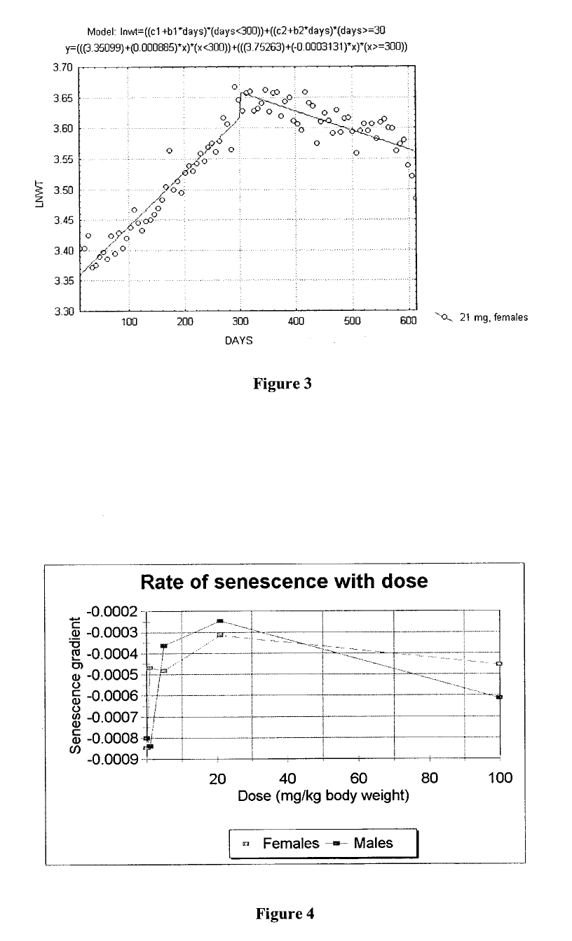

In both sexes, both strains and in all trials the relationship of mean body mass with age followed a two-phase relationship. After birth, this relationship has a positive gradient until they reached a critical age that is understood by the applicant to mark the onset of senescence. After this critical age, the rate of decline correlated with dose of pine bark extract. The higher the dose of pine bark extract, the slower the rate of senescence (see FIGS. 1, 2, 3 and 4 which show the rate of senescence corresponding to dose).

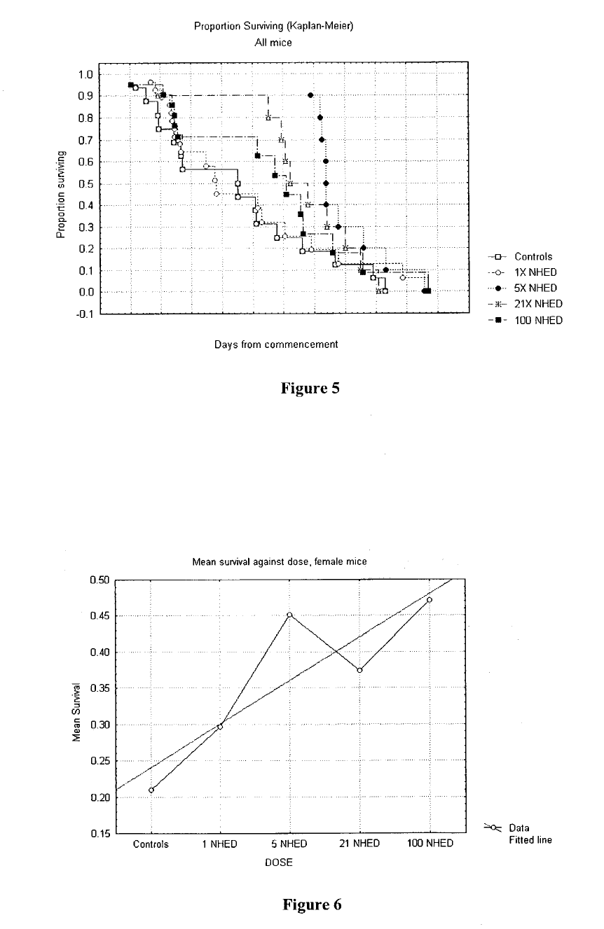

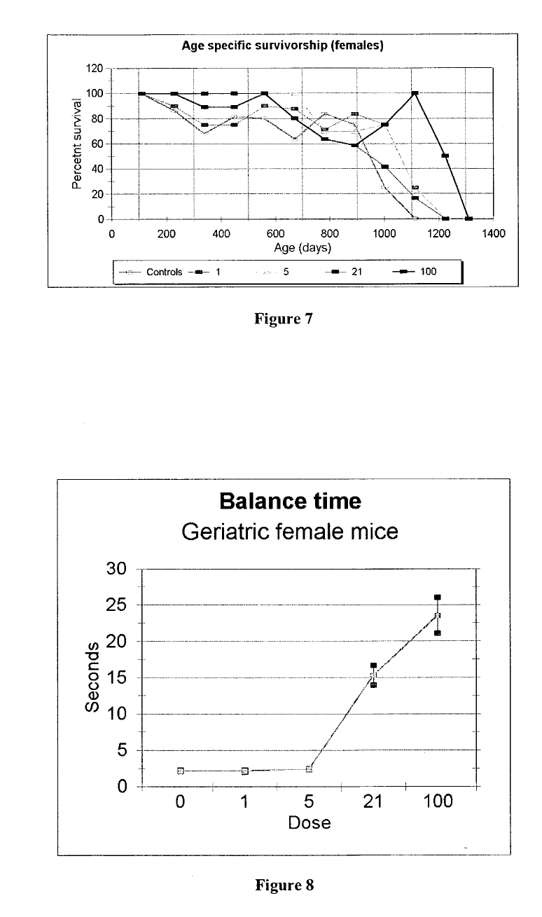

Referring to FIGS. 5, 6 and 7, it can be seen that an increase life span results from taking pine bark extract. The mice fed with pine bark extract dosages showed an increased proportion of survivorship (FIG. 5), a higher mean survival rate (FIG. 6) and a greater average life span (FIG. 7).

A further test was run towards the end of the study on the elderly mice. The analysis was done by repeated measure of the neuromuscular performance in very elderly female mice by measuring the time taken (duration) before a mouse placed on a wooden rod fell. The results of this analysis are shown in FIG. 8.

The results show that the time taken before a mouse placed on a wooden rod fell was strongly non-linear and positively related to dose i.e. mice on average were able to balance for approximately 6 times longer with a dose of 21 mg/kg and approximately 9 times longer for a dose of 100 mg/kg. The results thus show that pine bark extract slows down neuromuscular senescent decline in a dose related manner. That is, it reduces the rate of neuromuscular performance decline and hence in at least this symptom, decreases the rate of senescent decline.

Thus, the present invention contemplates providing a composition including a dose of flavonoids extracted from pine bark that has at least one mode of action selected from the group including: decreasing the rate of senescence of a mammal after onset of senescence; increasing the life span of a mammal; maintaining neuromuscular performance of a mammal after onset of senescence; increasing neuromuscular performance of a mammal after onset of senescence; and combinations thereof.

The above composition has been found by the applicant to be particularly advantageous in delaying the rate of senescence and its related symptoms in mammals. Trials completed by the applicant on mice indicate a reduction in the rate of senescence after onset by as much as 37% for mice fed with a regular dose of pine bark extract. This in itself has the implication of an improved quality of life for mammals after the onset of senescence.

Further results indicate an increase in life span by 8 to 17%. Further results also show that the proportion of mice surviving longer is 80% higher for those taking a regular dose of pine bark extract. This combination of results and the close phylogenetic relationship between mice and humans³⁰ show that this composition can be used to effectively increase the lifespan of a human.

While the present example has used pine bark extract as the main composition, it will be appreciated that other Pinus radiata compounds with similar properties may also be used.

While a particular embodiment of the Increased Lifespan Formulation has been described herein, it will be appreciated by those skilled in the art that changes and modifications may be made thereto without departing from the invention in its broader aspects and as set forth in the following claims.

### REFERENCES

1. Harman D. 1956. Aging—a theory based on free radical and radiation chemistry. Journal of Gerontology, 11:A614.

2. Harman D. 1981. The aging process. Proc. Natl. Acad. Sci. U.S.A.

3. Halliwell, B, Gutteridge, J M C. 1984. Oxygen toxicity, oxygen radicals, transition metals, and disease, Biochem. J. 219:1-14.

4. Halliwell, B, Gutteridge J M C. 1999. Free radicals in biology and medicine, (3^{rd}. ed.). Oxford: Oxford University Press.

5. Ames, B N, Shigenaga, M K, Hagen, T M. 1993. Oxidants, and the degenerative diseases of aging. Proc. Natl. Acad. Sci, U.S.A. 90:7915-7922.

6. Bland, J S 1995. Oxidants and antioxidants in clinical medicine: past, present, and future potential. J. Nutr. Env. Med., 5:255-280.

7. Diplock, A. T. 1994. Antioxidants and disease prevention. Mol. Aspects Med., 15:293 376.

8. Halliwell, B. 1996. Antioxidants in human health and disease. Ann. Rev. Nutr. 16: 33-50.

9. Beckman K B, Ames B N. 1998. The free radial theory of aging matures. Physiological Revues, 78:547-581.

10. Yan L -Y, Sohal R J. 2000. Prevention of flight activity prolongs the life span of the house fly, Musca domestica, and attenuates the age-associated oxidative damage to specific mitochondrial proteins. Free Radical Biology and Medicine, 29(11):1143 1150.

11. Cheesman, K H., Slatter, T F. 1993. An introduction to free radical biochemistry. Brit. Med. Bull., 49:481-493.

12. Gutteridge J M C, Halliwell B. 2000. Free radicals and antioxidants in the year 2000: a historic look to the future. In:Reactive Oxygen Species:From Radiation to Molecular Biology:a Festschrift in Honor of Daniel L. Gilbert. (Ed) Chuang Chin Chiueh. Ann N Y Acad Sci, 899:136-147.

13. Lindsay D G. 1999. Diet and ageing:the possible relation to reactive oxygen species. Journal Nutr Health Aging, 3(2):84-91.

14. Meydani M, et al. 1998. The effect of long-term dietary supplementation with antioxidants. Annals New York Academy of Science, 254:352-360.

15. Meydani M. 1999. Dietary antioxidants modulation of aging and immune endothelial cell interaction. Mech Ageing Dev, 111(2-3): 123-132.

16. Lipman R D, et al. 1998. Disease incidence and longevity are unaltered by dietary antioxidant supplementation initiated during middle age in C57BL/6 mice. Mechanisms of Ageing and Development, 103:269-284.

17. Bezlepkin, V. G., Sirota, N. P., Gaziev, A. I. 1996. The prolongation of survival in mice by dietary antioxidants depends on their age by the start of feeding this diet. Mechanisms of Aging and Development. 92:227-234.

18. Harris S B, Weindruch R, Smith G S, Mickey M R, Walford R L. 1990. Dietary restriction alone and in combination with oral ethoxyquin/2-mercaptoethylamine in mice. J Gerontology 45(5):B141-147.

19. Blackett A D, Hall D A. 1981. Vitamin E—its significance in mouse ageing. Age Ageing 10(3):191-195.

20. Massie H R, Aiello V R, Doherty T J. 1984. Dietary vitamin C improves the survival of mice. Gerontology 30(6):371-375.

21. Winter J C. 1984. The effects of an extract of Ginko biloba on cognitive behavior on the cognitive behavior and longevity in the rat. Physiol Behav 63:425-33.

22. Gallagher I M; Clow A; Glover V. 1998. Long term administration of(−) diprenyl increases mortality in male Wistar rats. J Neural Transmission Supplementum 52:315-320.

23. Armeni T, et al. 1997. Dietary restriction affects antioxidant levels in rat liver mitochondria during ageing. Mol Aspects Med 18 Suppl:S247-250.

24. Ishige, Kumiko; Schubert, David; Sagara, Yutaka. 2001. Flavonoids protect neuronal cells from oxidative stress by three distinct mechanisms. Free Radical Biology and Medicine, 30(4):433-466.

25. Sohal R S, Sohal B T, Brunk U T. Relationship between antioxidant defenses and longevity in different mammalian species. Mech Ageing Dev, 53:217-227.

26. Lim D S, et al. 2000. Analysis of ku80-mutant mice and cells with deficient levels of. Mol Cell Biol 20(11):3772-80. QH 506.M7186

27. Bafitas, H. Sargent, F. 1977. Human Physiological adaptability through the life sequence. J Gerontology,

28. Aragona M, Maisano R, Panetta S, Giudice A, Morelli M, Lla Torre I, La Torre F. 2000. Telomere length maintenance in aging and carcinogenesis. Int J Onch 17(5):981-9.

29. Puka A A, Daly M L, Brewster S J, Matise T C, Barrett J, Shea-Drinkwater M, Kang S, Joyce E, Nicoli J, Benson E, Kunkel L M, Perls T. 2001. A genome-side scan for linkage to human exceptional longevity identifies a locus on chromosome 4. Proc. Natl. Acad. Sci. USA 98(18):10505-10508.

30. ‘Of Mice and Men’ 2002. New Scientist, vol.176, No.2372, pp12-13.

31. Heidrick M L, Hendricks L C, Cook D E 1984. Effect of dietary 2-mercaptoethanol on the lifespan, immune system, tumour incidence and lipid peroxidation damage in spleen lymphcytes of aging BC3F mice. Mechanisms of Ageing & Development 27(9184) 341-358.

What is claimed:

1. A composition including a dose of flavonoids extracted from pine bark that has at least one mode of action selected from the group consisting of: decreasing the rate of senescence of a mammal after onset of senescence; increasing the life span of a mammal; maintaining neuromuscular performance of a mammal after onset of senescence; increasing neuromuscular performance of a mammal after onset of senescence; and combinations thereof.

2. The composition of claim 1 wherein the dose is delivered to the mammal daily.

3. The composition of claim 1 wherein the dose is from 0.5 to 100 mg/kg body mass of flavonoids per day.

4. The composition of claim 1 wherein the dose is 5 mg/kg of body mass per day.

5. The composition of claim 1 wherein the pine bark extract includes primarily flavonoid compounds and associated compounds found naturally in the extracts.

6. The composition of claim 1 wherein the pine bark extract exhibits antioxidant behavior in vivo.

7. The composition of claim 1 wherein the pine bark extract is sourced from the bark of Pinus radiata.

8. The composition of claim 1 wherein the pine bark extract is extracted using a water-based process.

9. The composition of claim 1 wherein the composition includes further anti-oxidant active components.

10. The composition of claim 1 wherein the composition is formulated using components selected from the group consisting of: fillers; excipients; modifiers; humectants; stabilizers; emulsifiers; and other known formulation components.

11. The composition of claim 1 wherein the composition is administered in a form selected from the group consisting of: a tablet; a capsule; a suppository; an injection; a suspension; a drink; a tonic; a syrup; a powder; an ingredient in solid foods; an ingredient in liquid foods; and combinations thereof.

12. The composition of claim 1 wherein the composition is administered orally as a powder mixed with food.

13. The use of a composition including a dose of flavonoids extracted from pine bark, wherein the composition is administered to a mammal to have at least one mode of action on the mammal selected from the group consisting of: reducing the rate of senescence of a mammal after onset of senescence; increasing the life span of a mammal; maintaining the neuromuscular performance of a mammal after the onset of senescence; increasing the neuromuscular performance of a mammal after the onset of senescence; and combinations thereof.

14. The use of a composition as claimed in claim 13 wherein the dose is delivered to the mammal daily.

15. The use of a composition as claimed in claim 13 wherein the dose is from 0.5 to 100 mg/kg body mass of flavonoids per day.

16. The use of a composition as claimed in claim 13 wherein the dose is 5 mg/kg of body mass per day.

17. The use of a composition as claimed in claim 13 wherein the pine bark extract includes primarily flavonoid compounds and associated compounds found naturally in the extracts.

18. The use of a composition as claimed in claim 13 wherein the pine bark extract exhibits antioxidant behavior in vivo.

19. The use of a composition as claimed in claim 13 wherein the pine bark extract is sourced from the bark of Pinus radiata.

20. The use of a composition as claimed in claim 13 wherein the pine bark extract is extracted using a water-based process.

21. The use of a composition as claimed in claim 13 wherein the composition includes further anti-oxidant active components.

22. The use of a composition as claimed in claim 13 wherein the composition is formulated using components selected from the group consisting of: fillers; excipients; modifiers; humectants; stabilizers; emulsifiers; and other known formulation components.

23. The use of a composition as claimed in claim 13 wherein the composition is administered in a form selected from the group consisting of: a tablet; a capsule; a suppository; an injection; a suspension; a drink; a tonic; a syrup; a powder; an ingredient in solid foods; an ingredient in liquid foods; and combinations thereof.

24. The use of a composition as claimed in claim 13 wherein the composition is administered orally as a powder mixed with food.

25. A method of treatment of a mammal by administration of a composition including a dose of flavonoids extracted from pine bark to a mammal, to have at least one mode of action on the mammal selected from the group consisting of:reducing the rate of senescence of a mammal after onset of senescence; to increase the life span of a mammal; maintaining the neuromuscular performance of a mammal after the onset of senescence; increasing the neuromuscular performance of a mammal after the onset of senescence; and combinations thereof.

26. The method of claim 25 wherein the dose is delivered to the mammal daily.

27. The method of claim 25 wherein the dose is from 0.5 to 100 mg/kg body mass of flavonoids per day.

28. The method of claim 25 wherein the dose is 5 mg/ikg of body mass per day.

29. The method of claim 25 wherein the pine bark extract includes primarily flavonoid compounds and associated compounds found naturally in the extracts.

30. The method of claim 25 wherein the pine bark extract exhibits antioxidant behavior in vivo.

31. The method of claim 25 wherein the pine bark extract is sourced from the bark of Pinus radiata.

32. The method of claim 25 wherein the pine bark extract is extracted using a water-based process.

33. The method of claim 25 wherein the composition includes further anti-oxidant active components.

34. The method of claim 25 wherein the composition is formulated using components selected from the group consisting of: fillers; excipients; modifiers; humectants; stabilizers; emulsifiers; and other known formulation components.

35. The method of claim 25 wherein the composition is administered in a form selected from the group consisting of: a tablet; a capsule; a suppository; an injection; a suspension; a drink; a tonic; a syrup; a powder; an ingredient in solid foods; an ingredient in liquid foods; and combinations thereof.

36. The method of claim 25 wherein the composition is administered orally as a powder mixed with food.
